(19) United States
(12) Patent Application Publication
(10) Pub. No.: US 2010/0249416 A1
(43) Pub. Date: Sep. 30, 2010

(54) CHEMICAL SYNTHESIS OF I-124BETA CIT IODINE-124 [2-BETA-CARBOMETHOXY-3BETA- (4. -IODOPHENYL) -TROPANE] FOR PET INVESTIGATIONS AND FOR RADIOTHERAPY

(75) Inventors: Domenico Martini, Roccafluvione (IT); Paola Panichelli, Civitanova Marche (IT); Gianluca Valentini, Macerata (IT)

Correspondence Address: EGBERT LAW OFFICES, 412 MAIN STREET, 7TH FLOOR, HOUSTON, TX, 77002, US

(73) Assignee: A.C.O.M ADVANCED CENTER ONCOLOGY MACERATA S.P.A., Montecosaro, (IT)

(21) Appl. No.: 12/682,431

(22) PCT Filed: Sep. 30, 2008
(86) PCT No.: PCT/EP2008/008272

§ 371 Date: Apr. 9, 2010

(30) Foreign Application Priority Data
Oct. 10, 2007 (IT) MC2007A000196

(51) Int. Cl. C07D 221/22

(52) U.S. Cl. 546/112

(57) ABSTRACT

The present invention concerns a new molecule, I-124βCIT Iodine-124[2β-carbomethoxy-3β-(4-iodophenyl)-tropane] and its process of synthesis, for use in PET investigations.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns a new molecule, I-124βCIT Iodine-124[2β-carbomethoxy-3β-(4-iodophenyl)-tropane] and its process of synthesis, for use in PET investigations.

2. Description of Related Art Including Information Disclosed Under 37 CFR 1.97 and 37 CFR 1.98.

PET (Positron Emission Tomography) is a diagnostic method through which the metabolism of the various organs to be examined may be studied, with a view to obtaining early diagnostic parameters for a variety of diseases. Over recent years, the use of this method has become very widespread in the USA and PET diagnostic centers are currently increasing all over Europe, mainly in the oncology sector. In this sector, PET technology introduces two clinical parameters, i.e. early diagnosis and therapy optimization, which change the life expectancy of patients and improve the management of their illness.

Other fields of application are becoming increasingly important, among which neurology, cardiology and rheumatology. In an era in which average life duration is continuously increasing, the role of PET is undoubtedly bound to assume major importance not only in the field of research into aging pathologies such as Parkinson's and Alzheimer's disease, but also in the studying of molecules through which early diagnoses of the onset of acute cardiac events—currently one of the main causes of death—can be made.

A PET analysis is performed by injecting a radiopharmaceutical into the patient and then following the distribution of this radiopharmaceutical inside the human body by means of a specific machine called a Positron Emission Tomography device.

The radiopharmaceuticals are composed of two essential parts, i.e. a radioisotope (which emits beta radiations) and a molecule which will bind with the radioisotope and which constitutes the metabolic substrate of the PET investigation. Radioisotopes are produced by an instrument called a Cyclotron and they are bound to the molecule to be examined by specific methods of chemical synthesis.

The radioisotope most commonly utilized today is 18-Fluoro which has a half-life of approximately two hours and which has chemical characteristics that enable it to bind easily, in liquid form, to various molecules.

18-Fluoro-deoxy glucose (FDG) is the molecule most commonly used and studied today, as may be observed from the following publications:
1. Kilbourn M R, Dence C S, Welch M J, Mathias C J. Fluorine-18 labeling of protein. J Nuvl Med 1987; 28: 462-70;
2. Schlyer, 2004. PET tracer and radiochemistry. Ann Acad Med Singapore; 33: 146-154;
3. Votaw J. R., Satter M. R., Nickles R J. Oxygen present during azeotropic drying drastically reduces the radiochemical yield of 2-FDG. J. Label. Compds. Radiopharm. 28, 83, 1990;
4. Nakao R., Kida T., Suzuki K., 2005. Factors affecting quality control of [¹⁸F]FDG injection: bacterial endotoxins test, aluminium ions test and HPLC analysis for FDG and CIDG. Applied Radiation and Isotopes 62: 889-895;
5. Fludeoxyglucose F 18 injection. In: The United States Pharmacopeia, 25th ed., and The National Formulary, 20th ed. Rockeville, Md.: United States Pharmacopeia Convention, Inc., 2002: 752-753;
6. Briner W H. USP monograph and tests of radiopharmaceutical purity [letter]. Am J Health Syst Pharm. 1995; 52: 1817-1818;
7. Capintec, Inc. [Pittsburg, Pa.], written communication, January 2002;
8. Fludeoxyglucose (¹⁸F) injection. In: European Pharmacopeia 4th ed Strasbourg, France: European Directorate for the Quality of Medicines; 2002: 2316-2319;
9. Wienhard K., Pawlik G., Nebeling B. J. Cereb. Blood. Flow Metab. 11,485, 1991;
10. 4, 7, 13, 16, 21, 24-Hexaoxa-1,10-diazobicyclo-(8,8,8)hexacosane (Kriptofix 222) [material safety data sheet]. St. Louis, Mo.: Sigma-Aldrich; Aug. 3, 2000;
11. Meyer G. J., Coenen H. H., Waters S. L., Luxen A., Maziere B., Langstom B. PET radiopharmaceuticals in Europe: current use and data relevant for the formulation of summaries of product characteristics (SPCs). Eur J Nucl Med 22,1420, 1995;
12. Bacterial endotoxins test (general charter 85). In: The United States Pharmacopeia, 25th ed., and The National Formulary, 20th ed. Rockville, Md.: United States Pharmacopeial Convention, Inc.; 2002: 1889-1893;
13. Finished drug product controls and acceptance criteria. In: Guidance: PET Drug Products—Current Good Manufacturing Practice (CGMP). Rockville, Md.: Food and Drug Administration; 2002:27-28;
14. D J Schlyer. PET tracers and radiochemistry. Ann Acad Med Singapore 2004; 33: 146-54;
15. Sanjiv Sam Gambhir. Molecular imaging of cancer with positron emission tomography. Nat Rev Cancer, 2002; September (2): 683-693);
16. Purnima Dubey, Helen Su, Nona Adonai, Shouying Du, Antonio Rosato, Jonathan Braun, Sanjiv S. Gambhir, Owen N. Witte; Quantitative imaging of the T cell antitumor response by positron-emission tomography. PNAS Feb. 4, 2004; 100(3): 1232-1237;
17. Czernin J. Clinical applications of FDG-PET in oncology. Acta Med Aust 2002; 29: 162-70;
18. W. B. Eubank and D. A. Mankoff, Evolving role of Positron Emission Tomography in breast cancer imaging. Semin Nucl Med 2005; 35: 84-99;
19. Peremans K, Cornelissen B, Van Den Bossche B et al. A review of small animal imaging planar and pinhole SPECT Γ camera imaging. Vet Rad Ultrasound 2005; 46(2): 162-170;
20. Paul D. Acton, Small animal imaging with high resolution single photon emission tomography. Nucl Med Biol 2003; 30: 889-895;
21. Laforest R., Sharp T. L., Engelbach J. A. et al. Measurement of input functions in rodents: challenges and solutions. Nucl Med Biol 2005; 32: 679-685;
22. C S Levin, E J Hoffman. Calculation of positron range and its effect on the fundamental limit of positron emission tomography system spatial resolution. Phys Med Biol 1999 mar; 44(3): 781-799);
23. Peremans K., Cornelissen B., Van Den Bossche B. et al. A review of small animal imaging planar and pinhole SPECT Γ camera imaging. Vet Rad Ultrasound 2005; 46 (2): 162-170;
24. Hiroshi Toyama, Masanori Ichise, Jeih-San Liow, Douglas C. Vines, et al. Evaluation of anesthesia effects on 18F-FDG uptake in mouse brain and heart using small animal PET. Nucl Med Biol 2004; 31: 251-256;
25. Jun G. Tjuvajev, Arjun Joshi, James Callegari, Laura Lindsley et al. A general approach to the non-invasive imaging of transgene using cis-linked herpes simplex virus thymidine kinase. Neoplasia October 1999; 1(4): 315-320.

The 18-Fluoro-deoxyglucose (FDG) molecule permits the identification within the body of sites presenting increased glucose consumption compared to normal metabolic standards, and hence possibly cancerous.

The limit that is emerging from this application is the insufficient specificity in differentiating tumoral sites from possible infection sites. In this context, experimentation has been carried out using other molecules, such as Fluorocholine, which increases diagnostic specificity, especially as regards certain forms of tumor, such as prostate, lung and brain tumors, as may be observed from the following publications:
1. ¹⁸F-Fluorocholine: A New Oncologic PET Tracer. The Journal of Nuclear Medicine Vol. 42 No. 12, December 2001;
2. Synthesis and Evaluation of ¹⁸F-Labeled Choline Analogs as Oncologic PET Tracers. The Journal of Nuclear Medicine Vol 42 No. 12, December 2001;
3. Localization of Primary Prostate Cancer with Dual-Phase ¹⁸F-Fluorocholine PET. The Journal of Nuclear Medicine Vol. 47 No. 2, February 2006;
4. Uptake of 18F-Fluorocholine, ¹⁸F-Fluoroethyl-L-Tyrosine, and 18F-FDG in Acute Cerebral Radiation Injury in the Rat: Implications for Separation of Radiation Necrosis from Tumor Recurrence. The Journal of Nuclear Medicine Vol. 45 No. 11, November 2004;
5. PET for Prostate Cancer Imaging: Still a Quandary or the Ultimate Solution? The Journal of Nuclear Medicine Vol. 43 No. 2, February 2002;
6. Development of ¹⁸F-Fluoroethylcholine for Cancer Imaging with PET: Synthesis, Biochemistry, and Prostate Cancer Imaging. The Journal of Nuclear Medicine Vol. 43 No. 2, February 2002;
7. Synthesis and Evaluation of ¹⁸F-labeled Choline as an Oncologic Tracer for Positron Emission Tomography Initial Findings in Prostate Cancer. Cancer Research Vol. 61, pagg. 110-117, Jan. 1, 2000;
8. Pharmacokinetics and Radiation Dosimetry of ¹⁸F-Fluorocholine. The Journal of Nuclear Medicine Vol. 43 No. 1, January 2002;
9. Positron Emission Tomography/Computed Tomography with F-18-fluorocholine for Restaging of Prostate of Prostate Cancer Patient: Meaningful at PSA<5 ng/ml? Molecular Imaging And Biology Vol. 8, pagg 43-48, 2006;
10. ¹⁸F-Labeled Bombesin Analogs for Targeting GRP Receptor-Expressing Prostate Cancer. The Journal of Nuclear Medicine Vol. 47 NO. 3, March 2006;

or Fluoro-thymidine, as shown in the References below:
1. 3′-¹⁸F-Fluoro-3′-Deoxy-L-Thymidine: A New Tracer for Staging Metastatic Melanoma? The Journal of Nuclear Medicine Vol. 44 No. 12, December 2003;
2. [¹⁸F]FLT PET for diagnosis and staging of thoracic tumors. European Journal of Nuclear Medicine and Molecular Imaging Vol. 30 No. 10, October 2003;
3. [¹⁸F]3-deoxy-3′:fluorothymidine positron emission tomography: alternative or diagnostic adjunct to 2-[¹⁸F]-fluoro-2-deoxy-D-glucose positron emission tomography in the workup of suspicious central focal lesions? The Journal of Thoracic and Cardiovascular Surgery Vol. 127 No. 4 Apr. 2004;
4. Potential impact of [¹⁸F]3′-deoxy-3′-fluorothymidine versus [¹⁸F]fluoro-2-deoxy-D-glucose in positron emission tomography for colorectal cancer. European Journal of Nuclear Medicine and Molecular Imaging Vol. 30 No. 7, July 2003;
5. Fully automated system of 3′-deoxy-3′-[¹⁸F]fluorothymidine. Nuclear of Medicine and Biology, August 2004 Vol. 31, 803-809;
6. Imaging Proliferation in Lung Tumors with PET: ¹⁸F-FLT Versus ¹⁸F-FD. The Journal of Nuclear Medicine Vol. 44 NO. 9, September 2003;
7. PET Imaging with ¹⁸F-FLT and Thymidine Analogs: Promise and Pitfalls. The Journal of Nuclear Medicine Vol. 44 NO. 9, September 2003;
8. Rat Studies Comparing ¹¹C-FMAU, ¹⁸F-FLT, and ⁷⁶Br-BFU as Proliferation Markers. The Journal of Nuclear Medicine Vol. 43 No. 12, December 2002;
9. Evaluation of 3′-Deoxy-3′-¹⁸F-Fluorothymidine for Monitoring Tumor Response to Radiotherapy and Photodynamic Therapy in Mice. The Journal of Nuclear Medicine Vol. 45 No. 10, October 2004;
10. ¹⁸F-Fluoro-L-Thymidine and ¹¹C-Methylmethionine as Markers of Increased Transport and Proliferation in Brain Tumors. The Journal of Nuclear Medicine Vol. 46 No. 12, December 2005;
11. Monitoring Tumor Cell Proliferation by Targeting DNA Synthetic Processes with Thymidine and Thymidine Analogs. The Journal of Nuclear Medicine Vol. 44 No. 12, December 2003;
12. In Vivo Validation of 3′deoxy-3′-[¹⁸F]fluorothymidine ([¹⁸F]FLT) as a Proliferation Imaging Tracer in Humans: Correlation of [¹⁸F]FLT Uptake by Positron Emission Tomography with Ki-67 Immunohistochemistry and Flow Cytometry in Human Lung Tumors. Clinical Cancer Research Vol. 8, pagg. 3315-3323, November 2002;
13. 3-Deoxy-3-[¹⁸F]Fluorothymidine-Positron Emission Tomography for Noninvasive Assessment of Proliferation in Pulmonary Nodules. Cancer Research Vol. 62, pagg. 3331-3334, Jun. 15, 2002;
14. Usefulness of 3′-[F-18]Fluoro-3′-deoxythymidine with Positron Emission Tomography in Predicting Breast Cancer Response to Therapy. Molecular Imaging and Biology Vol. 8, pagg. 36-42, 2002;
15. A simplified analysis off [¹⁸F]3′-deoxy-3′-fluorothymidine metabolism and retention. European Journal of Nuclear Medicine and Molecular Imaging Vol. 32 No. 11, November 2005;
16. [¹⁸F]3′-Fluorothymidine, a Much Promising New Oncological PET Tracer: From Precursor Synthesis to Pet Images. Regional workshop on F-18 radiopharmaceuticals, Smolenice, Slovakia, Nov. 25-27, 2001;
17. [¹⁸F]FLT-PET in oncology: current status and opportunities. European Journal of Nuclear Medicine and Molecular Imaging 23 Nov. 2004.

Another sector of great development in the field of PET diagnostics is that involving the development of radiotherapy treatment plans designed to optimize therapeutic effects. In this context, work is at various stages of development in the sector, spanning from the demonstrated usefulness of FDG to the recent experiments with the isotope copper-64, and the molecule ⁶⁴Cu-ATSM in particular, as may be observed from the following publications:
1. Molecular mechanism of copper uptake and distribution; Current Opinion in Chemical Biology 2002, 6: 171-180;
2. Biochemical Characterization of the Human Copper Transporter Ctrl; The Journal of Biological Chemistry, vol 277 No. 6, Issue of February 8, pp. 4380-4387, 2002;
3. A comparison of PET imaging characteristics of various copper radioisotopes; Eur J Nucl Med Mol Imaging (2005) 32: 1473-1480;
4. Copper Radionuclides and Radiopharmaceuticals in Nuclear Medicine; Nuclear Medicine and Biology, Vol 23, pp. 957-980, 1996;
5. Basic characterization of 64Cu-ATSM as a radiotherapy agent; Nuclear Medicine and Biology 32 (2005) 21-28;
6. Double-tracer autoradiography with Cu-ATSM/FDG and immunohistochemical interpretation in four different mouse implanted tumor models; Nuclear Medicine and Biology 33 (2006) 743-750;
7. A novel approach to overcome hypoxic tumor resistance: Cu-ATSM-guided intensity-modulated radiation therapy; Int. J. Radiation Oncology Biol. Phys. Vol 49, no 4, pp. 1171-1182, 2001;
8. Mouse Extrahepatic Hepatoma Detected on MicroPET Using Copper (II)-64 Chloride Uptake Mediated by Endogenous Mouse Copper Transporter 1; Mol Imaging Biol (2005) 7:325-329;
9. Copper-64-diacetyl-bis(N4-methylthiosemicarbazone): An agent for radiotherapy; PNAS 2001; 98; 1206-1211;
10. Enhancing Targeted Radiotherapy by Copper (II) diacetyl-bis(N4-methylthiosemicarbazone) Using 2-Deoxy-d-Glucose; Cancer Research 63, 5496-5504, Sep. 1, 2003;
11. Molecular imaging with copper-64; Journal of Inorganic Biochemistry 98 (2004) 1874-1901;
12. Theragnostic imaging for radiation oncology: dose painting by numbers; Lancet Oncol. 2005; 6:112-17;
13. Hypoxia imaging-directed radiation treatment planning; Eur J Nucl Med Mol Imaging (2006) 33: 44-53;
14. Subcellular Localization of Radiolabeled Somatostatin Analogues: Implications for Targeted Radiotherapy of Cancers; Cancer Research 63, 6864-6869, Oct. 15, 2003;
15. Three-dimensional maximum a posteriori (MAP) imaging with radiopharmaceutical labeled with three Cu radionuclides; Nuclear Medicine and Biology, Vol 33 (2006) 217-226;
16. Dosimetry of Internal Emitters; Journal of Nuclear Medicine Vol 46 No. 1 (Suppl) 18-27;
17. Dosimetry in Peptide Radionuclide Receptor Therapy; A Review; J Nucl. Med 2006, 47: 1467-1475;
18. Electron-and Positron-Emitting Radiolanthanides for Therapy: Aspects of Dosimetry and Production; J Nucl. Med. 2006, 47: 807-814;
19. Radiation-Induced Biologic Bystander Effect Elicited In Vitro by Targeted Radiopharmaceuticals Labeled with alpha-, beta-, and Auger Electron-Emitting Radionuclides; J Nucl. Med 2006, 47:1007-1015;
20. Auger Electron Spectra; Acta Oncologica Vol. 39, No 6, pp. 673-679, 2000.

This molecule enables a map of the distribution of oxygen to be obtained inside the tumor mass so that treatment plans may be modified both as regards the capacity to reduce the area of intervention and as regards the exclusion of areas that do not present oxygen and hence are unlikely to respond to radiotherapy.

Experiments of this type exist also with molecules marked with 18-Fluoro, such as F-MISO, as may be observed from the following publications:
1. Hypoxia and Glucose Metabolism in Malignant Tumors: Evaluation by [18F]Fluoromisonidazole and [18F]Fluorodeoxyglucose Positron Emission Tomography Imaging; Clinical Cancer Research Vol. 10, 2245-2252, Apr. 1, 2004;
2. Hypoxia-induced increase in FDG uptake in MCF7 cells; J Nucl Med 2001; 42:170-5; 3.
3. Effect of intratumoral heterogeneity in oxygenation status of FMISO PET, autoradiography and electrode P02 measurement in murine tumors; Int. J. Radiation Oncology Biol. Phys., Vol. 62, No. 3, pp. 854-861, 2005;
4. Effects of hypoxia on the uptake of tritiated thymidine, L-leucine, L-methionine and FDG in cultured cancer cells; J Nucl Med 1996; 37:502-6;
5. Biologic correlates of (18) fluorodeoxy glucose uptake in human breast cancer measured by Positron Emission Tomography; J Clin Oncol 2002; 20:379-87;
6. The Hypoxic cell: a target for selective cancer therapy; Cancer Res 1999; 59:5863-70;
7. Characterization of [18F]fluoroetanidazole, a new radiopharmaceutical for detecting tumor hypoxia; J Nucl Med 1999; 40:1072-9;
8. Prognostic impact of Hypoxia Imaging with 18F-Misonidazole PET in Non-Small Cell Lung Cancer and Head and Neck Cancer Before Radiotherapy; J Nucl Med 2005; 46:253-260;
9. Imaging oxygenation of human tumours; Eur Radiol (2007) 17: 861-872;
10. Assessment of Hypoxia and Perfusion in Human Brain Tumors Using PET with 18F-Fluoromisonidazole and ISO-H₂O; J Nucl Med 2004; 45: 1851-1859;
11. On Measuring Hypoxia in Individual Tumors with Radiolabeled Agents; J Nucl Med. Vol. 42 No. 11 Nov. 2001;
12. Introducing fluorine-18 fluoromisonidazole positron emission tomography for the localisation and quantification of pig liver hypoxia; Eur J Nucl Med 1999 26:95-109;
13. Fully automated one-pot synthesis of [18F]Fluoromisonidazole; Nucl Med and Biology 32 (2005) 553-558.

However, the characteristics of Copper-64 and, in particular, its half-life of approximately 12 hours, makes it a radioisotope of great prospective interest in the sector.

Iodine-124 (¹²⁴I), the subject of many publications:
1. PET quantitation and imaging of the non-pure positron-emitting iodine isotope 124I; Applied Radiation and Isotopes 56 (2002) 673-679;
2. Preparation of ¹²⁴I solutions after thermodistillation of irradiated ¹²⁴TeO2 targets; Applied Radiation and Isotopes 52 (2000) 181-184;
3. Highly sensitive spectrophotometry determination of trace amounts of tellurium(IV) with the tungstate-basic dyes-poly (vinyl alcohol) system; Analyst, April 1998, Vol. 123 (695-697);
4. Quantitation of small-animal ¹²⁴I activity distributions using a clinical PET/CT scanner; Nucl Med 2004; 45:1237-1244;
5. Performance of a block detector PET scanner in imaging non-pure positron emitters modelling and experimental validation with ¹²⁴I; Phys. Mod. Biol. 49 (2004) 5505-5528;
6. Synthesis and preliminary evaluation of L-O-[¹²³I]IODODOPA as a potential sped brainimaging agent; Journal of labelled compound and radiopharmaceuticals—Vol.)(XVIII, No. 2;
7. Iodine-124 labelled Annexin—V as a potential radiotracer to study apoptosis using positron emission tomography; Applied Radiation and Isotopes 58 (2003) 55-62;
8. Imaging apoptosis in vivo using 124I-annexin V and PET; Nuclear Medicine and Biology 32 (2005) 395-402;
9. [¹⁸F]β-CIT-FP is superior to [¹¹CJβ-CIT-FP for quantitation of the dopamine transporter; Nuclear Medicine & Biology, Vol. 24, pp. 621-627, 1997;
10. Evaluation of dosimetry of radioiodine therapy in benign and malignant thyroid by means of iodine-124 and PET; Eur. J. Nucl. Med. (2002) 29:760-767;
11. ¹²⁴I in PET imaging: impact on quantification, radiopharmaceutical development and distribution; Eur. J. Nucl. Med. (2006) 33:1247-1248;
12. Value of ²⁴¹I-PETZCT in staging of patients with differentiated thyroid cancer; Eur. Radiol. (2004) 14:2092-2098;

is an unstable isotope that is not present in nature with a half-life lasting the equivalent of 4.2 days. Iodine-124 (¹²⁴I) is produced through a nuclear reaction: ¹²⁴Te(p,n)¹²⁴I from ¹²⁴TeO with an isotopic purity more than 99.8% using protonic energy in a range of 14-10 MeV; this nuclear reaction generates a high degree of purity of ¹²⁴I, while ¹²⁵I and ¹²⁶I levels are inferior to 0.01%.

¹²⁴I is therefore an ideal isotope and it is used as a radiotracer in nuclear medicine for Positron Emission Tomography (PET).

For the production of the radioisotope Iodine-124 (¹²⁴I), we use a 18 MeV IBA cyclotron and a solid target (COSTIS) dedicated to the production of Cu 64 and Iodine 124.

The method involves bombardment with protons for approx. eight hours at a current of 18 μA, on enriched Tellurium oxide (Te(124)O₂), mass of Tellurium oxide 200 mg, on a platinum disc (target support).

The yield after bombardment is 40-50 mCi of Iodine-124 (¹²⁴I), the cross-section of the beam's energy is 14 Mev. Moreover, in the radiation of the solid targets, it is vital for the proton beam to be perfectly centered; to ensure this, it is indispensable to know its shape. This is detected by a special scanner for autoradiographs (Cyclone) which records the image of a previously radiated aluminum disc in a phosphorus film.

The support used for the Target is a Platinum disc with a diameter of 24 mm and a circular cavity of 12 mm which guarantees good conductivity and high resistance to corrosion.

A mixture of isotopically enriched tellurium oxide (¹²⁴TeO₂) together with aluminum oxide (Al₂O₃) is used, approx. 250 mg w/5%. The aluminum acts as a binder for the crystalline vitreous matrix.

The mixture is melted at 753 degrees celsius and allowed to re-solidify (preparation time=2 hours). All this takes place in a quartz furnace.

The target must be as stable as possible against the high bombarding currents in order to minimize any losses of TeO2, when the vapor voltage assumes significant values.

Once the target is ready, bombardment commences. The same disc may be subjected to several radiations.

The Iodine-124 (¹²⁴I) is separated from the matrix of the target disc (¹²⁴TeO₂/allumina) by means of a process of thermodistillation² carried out using the TERIMO-Automatic 124/123I iodine isotope synthesis module for PET scanning System control of 124I radioactive iodine isotope synthesis.

System control is based on a PLC, a temperature regulator, an air flow regulator and a Scada System, used for the purpose of controlling and acquiring data.

Iodine-124 (¹²⁴I) recovery time is approximately one hour, the mixture is melted at 753° C., the ¹²⁴I₂ is released under the form of gas, it is bubbled in a hyper-pure solution of NaOH 0.1 N; the ¹²⁴I₂ is trapped in the solution of NaOH under the form of NaI (sodium iodide at 95%), sodium iodate NaIO₃ and periodate NaIO₄ (5%).

The entire chemical process takes place inside a synthesis module located in a glove box, which guarantees the sterility of the product and its quality, inasmuch as it follows cGMP guidelines, guaranteeing a product the main characteristics of which are quality and efficacy.

At the end of the process, Na ¹²⁴I is obtained and can either be administered directly on its own as a radiotracer or, alternatively, used as a radio marker for the synthesis of new radiopharmaceuticals.

The radionuclidic purity of the ¹²⁴I is controlled by a germanium gamma spectrometer in order to detect the presence of iodine-125 [¹²⁵I], iodine-126[¹²⁶I], iodine-130[¹³⁰I] and iodine-131 [¹³¹I]. These impurities must be inferior to 0.1%.

Observing the energy peaks, the purity of the radionuclide must be superior to 99.5%.

The Iodine 124 (¹²⁴I) under the form of Na¹²⁴I is used for the PET analysis.

Its long half-life (4.18 days) enables both the development of multiple radiochemical syntheses and the detection of slow biochemical processes that could not be detected using tracers with a short half-life such as ¹¹C and ¹⁸F⁴.

The use of Iodine 124(¹²⁴I) is particularly indicated in radioimmunodiagnosis and in radioimmunotherapy as a dosimetry indicator in order to check the status of the therapy with ¹³¹I, used in the treatment of thyrotoxicosis and thyroid tumors.

Iodine 131(¹³¹I) has such high energy levels that they cannot be detected by the instrument; it cannot therefore be used for PET investigations. For this reason, it is used during radioimmunotherapy with (iodine-124) [¹²⁴I] as it enables the progress of the therapy to be followed through PET imaging.

The ¹²⁴I is a positron emitter with a relatively complex decay pattern and, given the fact that only 22% of the disintegrations refer to positrons, it was initially considered unsuitable for PET imaging studies.

In 1996, Pentlow demonstrated that even although ¹²⁴I emitted a low number of positrons, it was nonetheless suitable for the identification of tumors surrounded by a relatively low background activity, which is a typical feature of thyroid diseases.

Today, therefore, radiotherapy with iodine is a commonly accepted practice in the treatment of benign and malignant thyroid diseases. The dosimetry derives from PET data obtained approximately 1-13 days subsequent to the simultaneous oral administration of a therapeutic dose of Iodine 131(¹³¹I) and a diagnostic dose of ¹²⁴I.

The experimental protocol includes, more precisely, in the administering of 30 to 40 MBq (Iodine 124)¹²⁴I together with a therapeutic dose of Iodine 131(¹³¹I) of 526 to 1.237 MBq. Then, from the 5th to the 13th day after administration, four or five PET Scans are performed.

The first PET analysis takes place 24 hours after administration, the acquisition parameters are 10-15 minutes of emission and 2-3 minutes of transmission for the visual field. The PET analysis with (Iodine 124) ¹²⁴I therefore proves to be a suitable technique through which it is possible to study the kinetics of the iodine in the treatment of thyroid cancer.

There are two main advantages to be gained from the use of iodine-124 in nuclear medicine as opposed to the diagnostic application currently in use, i.e. iodine-123 with SPECT technique:

First, the possibility to quantitatively define the distribution of the tracer at basal nucleus level. This can only be performed in a semi-quantitative fashion with iodine-123 in SPECT.

Second, the possibility to follow over time, for a period of up to 4 days, the variations of the tracer inside the basal nuclei. Pharmacological tests may also be performed and effects assessed.

### BRIEF SUMMARY OF THE INVENTION

The present invention concerns a new molecule, I-124βCIT Iodine-124 [2β-carbomethoxy-3β-(4-iodophenyl)-tropane] and its process of synthesis.

Thanks to the use of iodine 124βCIT Iodine-124 [2β-carbomethoxy-3β-(4-iodophenyl)-tropane], presynaptic diagnostics relative to the activity of the striated bodies may be carried out in a quantitative manner—with the possibility to conduct pharmacological tests—and at a lower cost compared with iodine-123βCIT Iodine 123βCIT Iodine-124 [2β-carbomethoxy-3β-(4-iodophenyl)-tropane]; in other words, a marked increase in quality is obtained at a lesser cost.

### DETAILED DESCRIPTION OF THE INVENTION

To a 1 ml vial closed with a diffusion barrier and in inert atmosphere, we add, in the following order: 5 mCi of Na ¹²⁴I in a solution of 500 μl of NaOH 0.05 N; 50 μg of trialkylstanyl precursor ([2β-carbomethoxy-3β-(4-tributylstannylphenyl)tropane] or [2β-carbomethoxy-3β-(4-trimethylstannylphenyl)tropane]) dissolved, sonicating for 3 minutes, in 150 μl of ethanol; 50 μl Of H₃PO₄ 0.5 N; 50 μl Of CH₃CO₃H 0.02 M prepared at the moment of use by 100 μl 32% of peracetic acid dissolved in 2.4 ml of water. After 30 minutes at ambient temperature in an inert atmosphere, we add 100 μl NaHSO₃ in a solution prepared by dissolving 10 mg in 1 ml. We wait 5 minutes, after which we add 500 μl of a solution saturated with NaHCO₃, we transfer the activity onto a C18 Sep-Pak Light cartridge (preconditioned with 5 ml of ethanol and 5 ml of water for injectables) with a flow of 1 ml/min. We wash the C18 Sep-Pak Light cartridge with 20 ml of sterile water with a flow of 4 ml/min, and we elute the product with 7 ml of a solution at 50% of ethanol/sterile water with a flow of 1 ml/min, after which the product is passed through a 0.22 μm sterile filter and diluted with saline solution.

We claim:

1. Molecule of I-124βCIT Iodine-124[β-carbomethoxy-3β-(4-iodophenyl)-tropane], the two component synthesis of which is suitable for diagnostic treatment “in vivo” using PET technology.

2. Molecule of I-124βCIT Iodine-124[2β-carbomethoxy-3β-(4-iodophenyl)-tropane] as claimed in claim 1 characterized by the substitution of an alkyl tin group with Iodine-124 with a reaction at ambient temperature.

3. Process of synthesis of I-124βCIT Iodine-124 [2β-carbomethoxy-3β-(4-iodophenyl)-tropane] with the substitution of the alkyl tin group with Iodine-124 with a reaction at ambient temperature.

4. Molecule Iodine-124 [2β-carbomethoxy-3β-(4-iodophenyl)-tropane] with the formula:

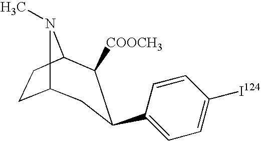

5. Process of synthesis of Iodine-124[2β-carbomethoxy-3β-(4-iodophenyl)-tropane] with the formula: [chemical structure]
